(19) United States
(12) Patent Application Publication
(10) Pub. No.: US 2017/0056104 A1
(43) Pub. Date: Mar. 2, 2017

(54) HEMODYNAMIC MEASUREMENT DEVICES AND TECHNIQUES

(71) Applicant: Mayo Foundation for Medical Education and Research, Rochester, MN (US)

(72) Inventors: Samuel J. Asirvatham, Rochester, MN (US); Amir Lerman, Rochester, MN (US)

(73) Assignee: Mayo Foundation for Medical Education and Research, Rochester, MN (US)

(21) Appl. No.: 15/120,276

(22) PCT Filed: Mar. 6, 2015
(86) PCT No.: PCT/US15/19117

§ 371 Date: Aug. 19, 2016

Related U.S. Application Data
(60) Provisional application No. 61/950,302, filed on Mar. 10, 2014.

(51) Int. Cl. A61B 18/14

(52) U.S. Cl. CPC A61B 18/1492; A61B 2018/0016

(57) ABSTRACT

Devices and methods are provided that can enhance the efficacy of medical treatments that cause hemodynamic effects. For example, this document provides devices and methods for enhancing the efficacy of medical treatments such as renal denervation and embolic protection procedures.

### CROSS-REFERENCE TO RELATED APPLICATIONS

This application claims the benefit of U.S. Provisional Application Ser. No. 61/950,302, filed Mar. 10, 2014. The disclosure of the prior application is considered part of (and is incorporated by reference in) the disclosure of this application.

### BACKGROUND

1. Technical Field

This document relates to devices and methods that can enhance the efficacy of certain medical treatments that cause hemodynamic effects. For example, this document relates to devices and methods for enhancing the efficacy of renal denervation and embolic protection procedures.

2. Background Information

Fractional flow reserve (FFR) is a physiological index that quantifies the severity of blood flow blockages in the coronary arteries. FFR measurement involves using pressure transducers to determine the ratio between the maximum achievable blood flow in a diseased coronary artery and the theoretical maximum flow in a normal coronary artery. An FFR of 1.0 is considered normal. An FFR lower than 0.75-0.80 is often considered indicative of a potential for myocardial ischemia.

FFR can be measured percutaneously using a pressure wire and calculating the ratio between coronary pressure distal to a coronary artery stenosis and aortic pressure under conditions of maximum myocardial hyperemia. The use of FFR is one example of how hemodynamic measurements can be used to enhance the efficacy of medical treatments.

### SUMMARY

This document provides devices and methods that can enhance the efficacy of certain medical treatments that cause hemodynamic effects. For example, this document provides devices and methods for enhancing the efficacy of medical treatments such as renal denervation and embolic protection procedures.

In general, one aspect of this document features a transcatheter deliverable medical device system. The transcatheter deliverable medical device system includes a delivery sheath, a mapping and ablation energy delivery device, and one or more pressure wires. The mapping and ablation energy delivery device that can be disposed on the distal end portion of a catheter. The mapping and ablation energy delivery device can be configured to expand to make contact with a vascular tissue of a patient when the mapping and ablation energy delivery device is not constrained by the delivery sheath. The one or more pressure wires can be configured to extend from the delivery sheath and distally beyond the mapping and ablation energy delivery device such that the one or more pressure wires can be placed within one or more sub-branches of the vascular tissue. The one or more pressure wires can each include one or more pressure transducers that are configured to provide a signal indicative of a blood pressure at the location of the one or more pressure transducers.

In various implementations of the transcatheter deliverable medical device system, the mapping and ablation energy delivery device may comprise a self-expandable member and one or more electrodes. Optionally, the mapping and ablation energy delivery device may comprise a nitinol member and a plurality of electrodes. Additionally, the mapping and ablation energy delivery device may comprise a balloon member and a plurality of electrodes in some implementations.

In a second general aspect, this document features a method for performing a renal denervation procedure. The method comprises delivering, to a renal artery, a transcatheter deliverable medical device system; measuring, by the one or more pressure transducers, a baseline blood pressure; delivering, by the mapping and ablation energy delivery device, stimulation energy to the renal artery; measuring, by the one or more pressure transducers, a post-stimulation blood pressure; comparing the baseline blood pressure to the post-stimulation blood pressure; and delivering, by the mapping and ablation energy delivery device and based on the comparison of the baseline blood pressure to the post-stimulation blood pressure, ablation energy to the renal artery. The transcatheter deliverable medical device system can include a delivery sheath, a mapping and ablation energy delivery device, and one or more pressure wires. The mapping and ablation energy delivery device that can be disposed on the distal end portion of a catheter. The mapping and ablation energy delivery device can be configured to expand to make contact with a vascular tissue of a patient when the mapping and ablation energy delivery device is not constrained by the delivery sheath. The one or more pressure wires can be configured to extend from the delivery sheath and distally beyond the mapping and ablation energy delivery device such that the one or more pressure wires can be placed within one or more sub-branches of the vascular tissue. The one or more pressure wires can each include one or more pressure transducers that are configured to provide a signal indicative of a blood pressure at the location of the one or more pressure transducers.

In various implementations of the method for performing a renal denervation procedure, the baseline blood pressure may be lower than the post-stimulation blood pressure. Optionally, the method may further comprise after delivering the ablation energy: measuring, by the one or more pressure transducers, a post-ablation blood pressure; delivering, by the mapping and ablation energy delivery device, additional stimulation energy to the renal artery; measuring, by the one or more pressure transducers, a post-ablation post-stimulation blood pressure; comparing the post-ablation blood pressure to the post-ablation post-stimulation blood pressure; and delivering, by the mapping and ablation energy delivery device and based on the comparison of the baseline blood pressure to the post-stimulation blood pressure, additional ablation energy to the renal artery.

In some embodiments of the method for performing a renal denervation procedure, the comparison of the post-ablation blood pressure to the post-ablation post-stimulation blood pressure indicates that the additional ablation energy can be effective for reducing the post-ablation post-stimulation blood pressure further.

In a third general aspect, this document features a method for monitoring for emboli. The method comprises: delivering to a cerebral vasculature of a patient one or more pressure-sensing wires that each include one or more pressure transducers that are each configured to provide a signal indicative of a blood pressure at the location of the one or more pressure transducers; measuring, by the one or more pressure transducers, a baseline blood pressure; installing an implantable medical device within the patient's vasculature; measuring, by the one or more pressure transducers and during or after the installing an implantable medical device within the vasculature of the patient, an intra-procedure blood pressure; and comparing the baseline blood pressure to the intra-procedure blood pressure to determine whether intravascular emboli have been generated by the installing of the implantable medical device.

In various implementations of the method for monitoring for emboli, the method may further comprise, in response to determining that intravascular emboli have been generated by the installing of the implantable medical device, installing or repositioning an emboli protection device within the patient's vasculature.

Particular embodiments of the subject matter described in this document can be implemented to realize one or more of the following advantages. In some apparatus embodiments provided herein, a pacing/ablation device and a pressure-sensing wire are synergistically combined. Such a combined apparatus can be used to measure intravascular blood pressure as part of a denervation procedure (such as a renal denervation procedure), to enhance the efficacy thereof Some embodiments of the pressure-sensing wires provided herein, can be used to enhance emboli detection techniques. Such techniques can be beneficially used in conjunction with stent placement procedures, for example. In some embodiments, the systems and methods provided herein can be used to treat patients in a minimally invasive fashion. Such minimally invasive techniques can reduce recovery times, patient discomfort, and treatment costs.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is an illustration of a renal denervation treatment technique using an ablation system including pressure-sensing wires in accordance with some embodiments provided herein.

FIG. 2 is a flowchart of a method for performing renal denervation using an ablation system including pressure-sensing wires in accordance with some embodiments provided herein.

FIG. 3 is an embodiment of a pressure-sensing wire in accordance with some embodiments provided herein.

FIG. 4 is another embodiment of pressure-sensing wire in accordance with some embodiments provided herein.

FIG. 5 is an embodiment of an ablation system including a pressure-sensing wire in accordance with some embodiments provided herein.

FIG. 6A is another embodiment of an ablation system including a pressure-sensing wire in accordance with some embodiments provided herein.

FIG. 6B shows the ablation system of FIG. 6A in an expanded configuration in accordance with some embodiments provided herein.

FIG. 7A is another embodiment of an ablation system including a pressure-sensing wire in accordance with some embodiments provided herein.

FIG. 7B is the ablation system of FIG. 7A with the ablative member shown in an expanded configuration in accordance with some embodiments provided herein.

Like reference numbers represent corresponding parts throughout.

### DETAILED DESCRIPTION

This document provides systems and methods that can enhance the efficacy of some medical treatments that cause hemodynamic effects. For example, this document provides systems and methods for enhancing the efficacy of medical treatments such as renal denervation and embolic protection procedures.

In the context of renal denervation, for example, the devices for vascular pressure sensing provided herein can be used to confirm whether sympathetic renal nerves have been successfully located and subsequently denervated. For example, as will be described more fully below, in some circumstances a pressure wire in accordance with some embodiments provided herein can be positioned in the renal vascular bed prior to denervation. A baseline blood pressure reading can be taken. Next, stimulation from a mapping/ablating device can be delivered in effort to stimulate sympathetic renal nerves. If the pressure wire does not detect an increase in blood pressure after the stimulation, it can indicate that the sympathetic renal nerves were not stimulated by the mapping/ablating device. However, if the pressure wire detects an increase in blood pressure within the renal vascular bed, it can be reasonably concluded that the mapping/ablating device is properly positioned to successfully ablate the sympathetic renal nerves, and such ablation can then be performed. In this manner, for example, the devices and methods provided herein can enhance a renal denervation treatment.

In the context of embolic-related protection procedures, as another example, the devices for vascular pressure sensing provided herein can be used to confirm whether emboli are being generated, and/or whether emboli protection techniques are being performed effectively. Embolic protection devices are sometimes used to capture particles (e.g., plaque) that may become dislodged during an interventional procedure. Embolic debris may flow downstream in the patient's vasculature and block smaller vessels, resulting in procedural complications or poor patient outcomes. Using a stent placement procedure (e.g., a coronary artery stent, a carotid artery stent, etc.) as an example, in some circumstances a pressure wire in accordance with some embodiments provided herein can be positioned in the cerebral vasculature, for example. A baseline blood pressure measurement can be taken. During the interventional procedure, if changes from the baseline blood pressure are detected, the changes may be interpreted as indicative of embolic occlusions within the cerebral vasculature. In such a case, an embolic protection device can be installed, or a previously installed embolic protection device can be repositioned or replaced in effort to enhance its efficacy.

It should be understood that the examples provided herein are illustrative and not exhaustive. Therefore, other implementations of the devices and techniques provided herein are also envisioned within the scope of this document. For example, other implementations would include, but are not limited to, the use of the FFR criteria with ganglia ablation or stimulation done in the aortic, celiac, or hepatic region.

In another example with reference to pseudo nerve activity, the devices and techniques provided herein use can be used in accordance with the following. Along with FFR, the blood pressure, peripheral vascular tone, and pseudo nerve activity can be simultaneously monitored and measured. This will facilitate the set up a template for FFR patterns when there are actual effects on the sympathetic output in each of these situations. Once the template is established, then this relatively noninvasive technique can be used to continue to monitor energy delivery and subsequent follow-up. In addition, responses seen in the FFR template can be assessed with other manipulations, such as the use of a pharmacological agent or stimulation or ablation of the ganglia elsewhere.

For some of the applications, it may be more meaningful to either perform the FFR in the venous anatomy standalone or compare results of FFR with the arterial anatomy. This comparison of fractional flow can be the input to drive the algorithm for therapy (the therapy itself may vary with the target organ) to assess completeness of renal denervation or stimulation.

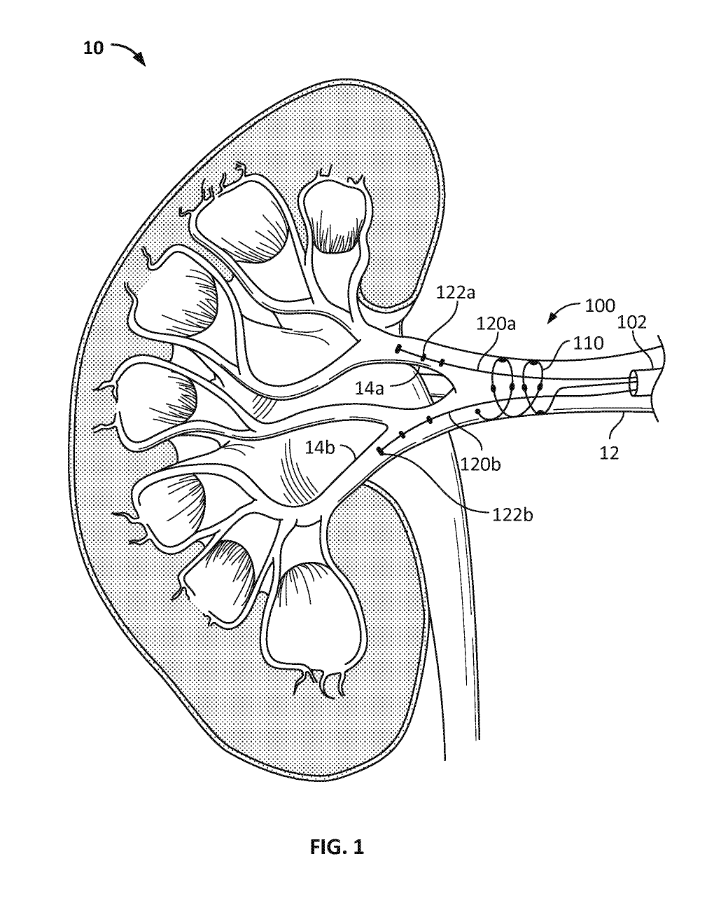

With reference to FIG. 1, a kidney 10 can undergo a renal denervation treatment using an example renal denervation system 100 in accordance with some embodiments provided herein. In the depicted embodiment, renal denervation system 100 includes an expandable member with integral mapping/ablation electrodes 110 (hereinafter referred to as ablation device 110), two pressure-sensing wires 120a and 120b, and a delivery sheath 102.

Renal denervation system 100 is delivered into a main renal artery 12 through delivery sheath 102. Upon emergence from delivery sheath 102, ablation device 110 expands to at least partially abut the inner wall of main renal artery 12. Mapping and ablation energy can be delivered from ablation device 110 to the inner wall of main renal artery 12. In the depicted embodiment, the ablation energy is electrical energy, but alternatively, other types of ablation energy sources can be used. Such ablation energy sources can include, but are not limited to radio-frequency, cryogenic, ultrasound, chemical, thermal, and the like, and combinations thereof.

In the depicted embodiment, ablation device 110 is a generally spiral structure that is constructed of a shape-memory material such as, but not limited to, nitinol. Alternatively, ablation device 110 can be another type of structure. Such alternative structures for ablation device 110 can include, but are not limited to, stent-like framework scaffolds, baskets, balloons, and the like, and combinations thereof.

Pressure-sensing wires 120a and 120b are also made to emerge from delivery sheath 102. Pressure-sensing wires 120a and 120b extend from delivery sheath 102 and are positioned beyond main renal artery 12 in the branches of the renal artery 14a and 14b. In these positions, the blood pressure within the branches of the renal artery 14a and 14b can be measured. In some embodiments, pressure-sensing wires 120a and 120b include piezoresistive types of pressure transducers. Other types of pressure transducers may also be used including, but not limited to, capacitive, piezoelectric, optical, potentiometric, resonant, and the like. Pressure-sensing wires 120a and 120b can be configured for placement in very small vessels. For example, in some implementations the vessels can range anywhere from about 1 mm up to about 6 cm. Thus, these could be used in the renal vessels themselves, in the aorta, in the veins, peripheral branches of the renal veins, or the inferior vena cava and elsewhere in specific circumstances such as the hemiazygos vein.

While the depicted embodiment of renal denervation system 100 includes two pressure-sensing wires 120a and 120b, in alternative embodiments one, three, or more than three pressure-sensing wires can be included.

Pressure-sensing wires 120a and 120b each include one or more pressure transducers 122a and 112b respectively. In this embodiment, each pressure-sensing wire 120a and 120b includes three pressure transducers, but alternatively one, two, four, or more than four pressure transducers may be included on other embodiments of pressure sensing wires within the scope of this disclosure. In some embodiments having multiple pressure transducers on a single pressure-sensing wire 120a and/or 120b, one or more of the pressure transducers 122a and/or 122b are movable in relation to one or more others of the pressure transducers 122a and/or 122b.

While the preceding example has presented inventive concepts in the context of renal denervation, the concepts are not limited thereto. For example, in some embodiments pulmonary arterial or branch artery flow reserve may be very useful as part of a feedback algorithm when denervating or doing ganglia ablation for pulmonary hypertension. Moreover, the flow can be compared in the aorta or a branch to see if effects on systemic hypertension are also being made.

Further, the concepts disclosed herein may be used for hepatic ganglia ablation to targets glucose intolerance and metabolic disturbances, for example. Such conditions are difficult to provide immediate feedback for. However, looking at the hepatic vasculature with FFR can be an effective way to do this.

Although flow is essentially what is used when calculating flow proportion or flow reserve, for some of the applications (particularly in the pulmonary artery or renal artery), the velocity itself or velocity along with pressure may produce a fractional measurement (comparison to max velocity or baseline pressure either standalone or as an adjunct to flow reserve) to make the best predictions on completeness or efficacy of an intervention.

Vagal nerve stimulation by the devices and techniques provided herein can be used as an investigative therapy for systolic heart failure. One implementation would be to look at flow reserve in either the gastric vessels or potentially even the coronary arteries. However, indwelling pressure sensors in the arterial system are problematic and at best could only be used when optimizing vagal stimulation algorithm at the time of implant. However, a pressure wire in the coronary venous system could potentially be kept in place. Since such patients do usually have a left ventricular pacing lead placed through the coronary sinus, one option is to integrate the pressure sensor to a pacing lead and use this as a feedback not only for the vagal nerve stimulation but the type of biventricular stimulation using the left ventricular lead itself.

Another advantageous use of an indwelling venous flow reserve measurement device, is to help with biofeedback. For example, the information can be fed back in a wireless manner to some handheld device, watch, etc., to signal optimization of flow in correlation with a particular activity to optimize training and to provide positive feedback.

Additionally, since the devices and methods provided herein allow velocity reserve to be used in addition to, or as an alternative to, flow, tissue velocity reserve is a novel concept herein. The concepts are akin and analogous to fractional flow reserve, but instead of flow, using velocity, and instead of red blood cell velocity, using tissue velocity. Optimization therapy, particularly vagal stimulators, spinal nerve stimulators, or carotid body stimulators, and looking at tissue velocity can be very beneficial. Taking this further, the devices and methods provided herein can be used to simultaneously look at flow reserve when placing a probe in the artery and tissue velocity reserve of the arterial wall itself as an ideal monitoring tool for the effectiveness and completeness of renal denervation.

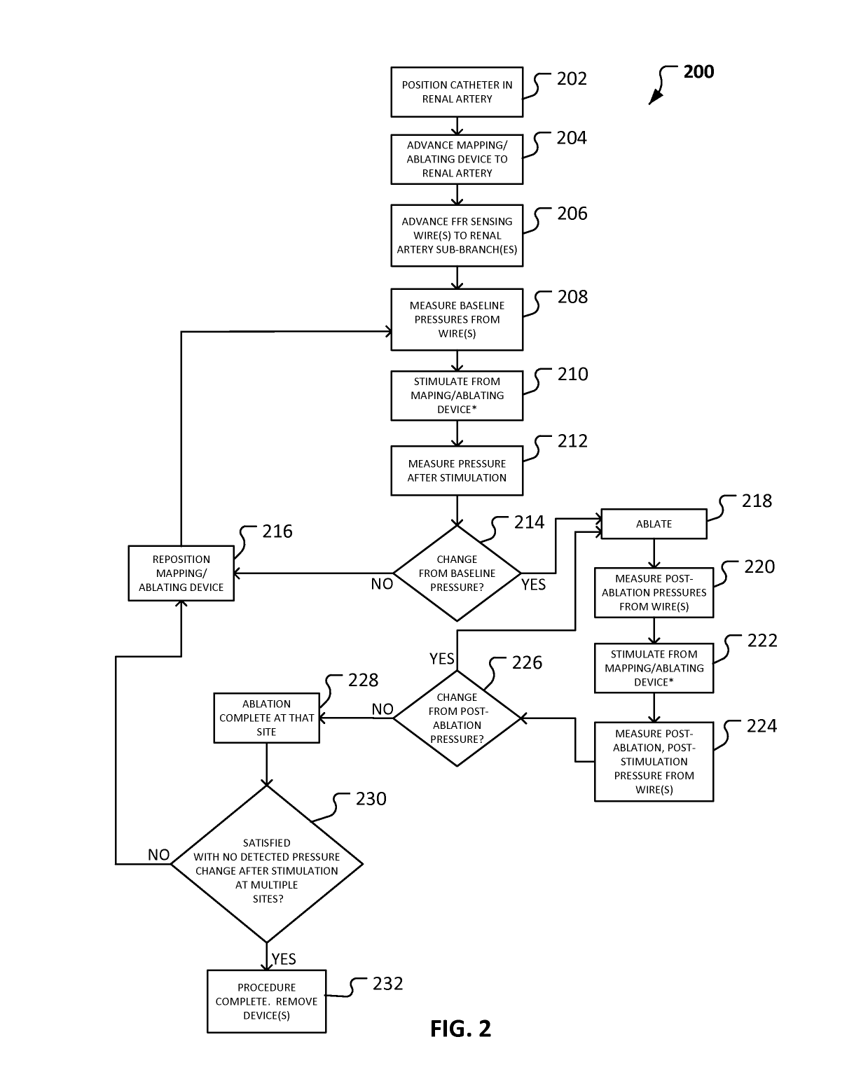

With reference to FIG. 2, a flowchart is provided that describes a method 200 for performing renal denervation using a combined mapping/ablation system with pressure-sensing wires. It should be understood that, while method 200 is described in the context of renal denervation, such techniques may also be implemented in the context of other denervation procedures without departing from the scope of this disclosure.

In some circumstances, method 200 can be used to provide confirmation of effective denervation of the sympathetic renal nerves, as will be explained. The ablation system used to perform method 200 can be configured such as renal denervation system 100 described above. However, other renal denervation system embodiments may also be used to perform method 200 without departing from the scope of this disclosure.

At step 202, a delivery sheath/catheter is positioned in the renal artery. Femoral access may be used, for example. At step 204, the mapping/ablative member of the ablation system is positioned within the renal artery such that the mapping/ablative member makes contact with the inner wall of the renal artery.

At step 206, one or more pressure-sensing wires are advanced from the delivery sheath to extend beyond the mapping/ablative member. The pressure transducer(s) of the pressure-sensing wire(s) are positioned in the sub-branches of the renal artery. At step 208, baseline blood pressure readings are taken by the pressure transducer(s) of the pressure-sensing wire(s).

At step 210, energy is delivered from the mapping/ablative member to stimulate the sympathetic renal nerves of the renal artery. The energy may or may not stimulate the sympathetic renal nerves, depending on the position of the sympathetic renal nerves in relation to the location of the energy delivery. If the delivery of the stimulation in step 210 results in actual stimulation of the sympathetic renal nerves, the renal vasculature will tend to constrict and the blood pressure therein will tend to rise. Conversely, if the delivery of the stimulation in step 210 does not result in actual stimulation of the renal artery, the renal vasculature will not tend to constrict and the blood pressure therein will remain stable. At step 212, the pressure-sensing wire(s) are used to measure blood pressure within the renal vasculature after the stimulation delivered in step 210. In some embodiments of method 200, step 210 can include administration of adenosine or capsaicin to cause further vasoconstriction, thereby amplifying the potential blood pressure change resulting from the stimulation.

At step 214, the blood pressures measured at steps 208 (baseline) and 212 (after delivery of stimulation) are compared. If no substantial change is detected, the method 200 proceeds to step 216 where the mapping/ablative member is repositioned within the renal artery. The repositioning is performed because the lack of change in the blood pressure readings can indicate that the mapping/ablative member is not positioned within the renal artery so as to affect the sympathetic renal nerves as desired. After the repositioning, step 208 and the steps thereafter are repeated. However, if a change is detected in step 214, the method 200 then proceeds to step 218.

At step 218, ablation energy is delivered from the mapping/ablative member to the renal artery. It can be reasonably anticipated that such an ablation energy delivery will be effective for denervating the sympathetic renal nerves because the detected change in the blood pressure readings indicated that the mapping/ablative member was in a position to do so.

At step 220, the post-ablation blood pressure is measured by the pressure-sensing wire(s). Step 220 can be considered largely analogous to step 208. At step 222, energy is delivered from the mapping/ablative member to the stimulate the renal artery again. The energy may or may not stimulate the sympathetic renal nerves, depending on how substantially the nerves in the effect area were denervated in step 218. If the delivery of the stimulation in step 222 results in actual stimulation of the renal artery, the renal vasculature will tend to constrict and the blood pressure therein will rise. Conversely, if the delivery of the stimulation in step 222 does not result in actual stimulation of the renal artery, the renal vasculature will not tend to constrict and the blood pressure therein will remain stable. At step 224, the pressure-sensing wire(s) are used to measure blood pressure within the renal vasculature after the stimulation of step 222. In some embodiments of method 200, step 224 can include administration of adenosine or capsaicin to cause further vasoconstriction, thereby amplifying the potential blood pressure change resulting from the stimulation.

In step 226, the blood pressures measured at steps 220 (post-ablation baseline) and 224 (after delivery of additional stimulation) are compared. If no substantial change is detected, the method 200 proceeds to step 228 because the ablation is considered to be substantially completed at that particular position of the mapping/ablative member within the renal artery. However, if a change is detected in step 226, the method 200 then reverts to step 218 where additional ablation energy is delivered without repositioning the mapping/ablative member.

Once ablation is complete at a particular position of the mapping/ablative member (as indicated in step 228), the method 200 proceeds to step 230. At step 230, the decision posed is whether the blood pressure as measured by the pressure-sensing wire(s) is sufficiently satisfactory to end the ablation procedure. If that is the case, method 200 then proceeds to step 232 and the ablation system is removed from the patient, ending the method 200. However, if in step 230 the blood pressure as measured by the pressure-sensing wire(s) is determined to be unsatisfactory, then the method 200 reverts to step 216 where the mapping/ablative member is repositioned. The repositioning of step 216 is performed because the lack of satisfactory change in the blood pressure readings can indicate that the ablation procedure has not yet resulted in the desired efficacy. After the repositioning of step 216, method 200 reverts to step 208. Step 208, and the steps thereafter, are subsequently repeated as described above.

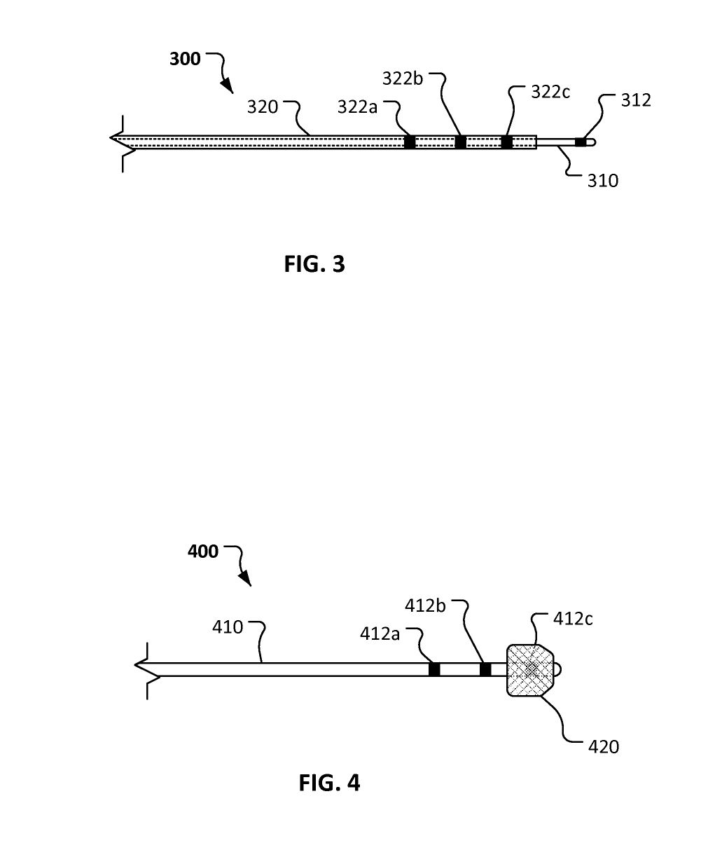

With reference to FIG. 3, an example pressure-sensing device 300 can include a guidewire 310 and a catheter 320. Catheter 320 can be configured for an over-the-wire arrangement with guidewire 310. In other words, catheter 320 can be slidable in relation to guidewire 310 in some embodiments. The ability to slide catheter 320 can allow a user to readily position catheter 320 as desired, and can allow the user to conveniently reposition catheter 320, in situ, as desired.

Catheter 320 can include one or more pressure sensors. In the depicted embodiment, catheter 320 includes three pressure sensors 322a, 322b, and 322c. In other embodiments, catheter 320 can include one, two, four, or more than four pressure sensors. The pressure sensors 322a, 322b, and 322c can be spaced apart from each other by various distances. For example, in some embodiments pressure sensors 322a, 322b, and 322c are spaced about 1 cm apart from each other. However, smaller or larger spacing is also envisioned within the scope of this disclosure.

Guidewire 310 can include one or more pressure sensors. In the depicted embodiment, guidewire 310 includes one pressure sensor 312. In other embodiments, guidewire 312 can include two or more pressure sensors, or may not include any pressure sensors in some other embodiments.

With reference to FIG. 4, an example pressure-sensing wire 400 includes a wire 410 and a spacing member 420. Spacing member 420 is disposed around wire 410.

Wire 410 can include one or more pressure sensors. In the depicted embodiment, wire includes three pressure sensors 412a, 412b, and 412c. In other embodiments, wire 410 can include one, two, four, or more than four pressure sensors. The pressure sensors 412a, 412b, and 412c can be spaced apart from each other by various distances. For example, in some embodiments pressure sensors 412a, 412b, and 412c are spaced about 1 cm apart from each other. However, smaller or larger spacing is also envisioned.

Spacing member 420 is disposed around the periphery of wire 410 so as to protect pressure sensors 412a, 412b, and 412c from becoming wedged into a vessel, which could result in false pressure readings. In the depicted embodiment, spacing member 420 is a stent-like frame that can be constructed, for example, of nitinol. In some implementations, pressure-sensing wire 400 is delivered using a delivery sheath and spacing member 420 is collapsed in a low profile when it is contained within the sheath. Upon emergence from the sheath in situ, spacing member 420 can self-expand to a larger profile such that the outer periphery of spacing member 420 is spaced away from the outer diameter of wire 410. In alternative embodiments, other types of spacing members can be used, such as, but not limited to, balloon(s), spiral members, fabrics, elastomers, and the like.

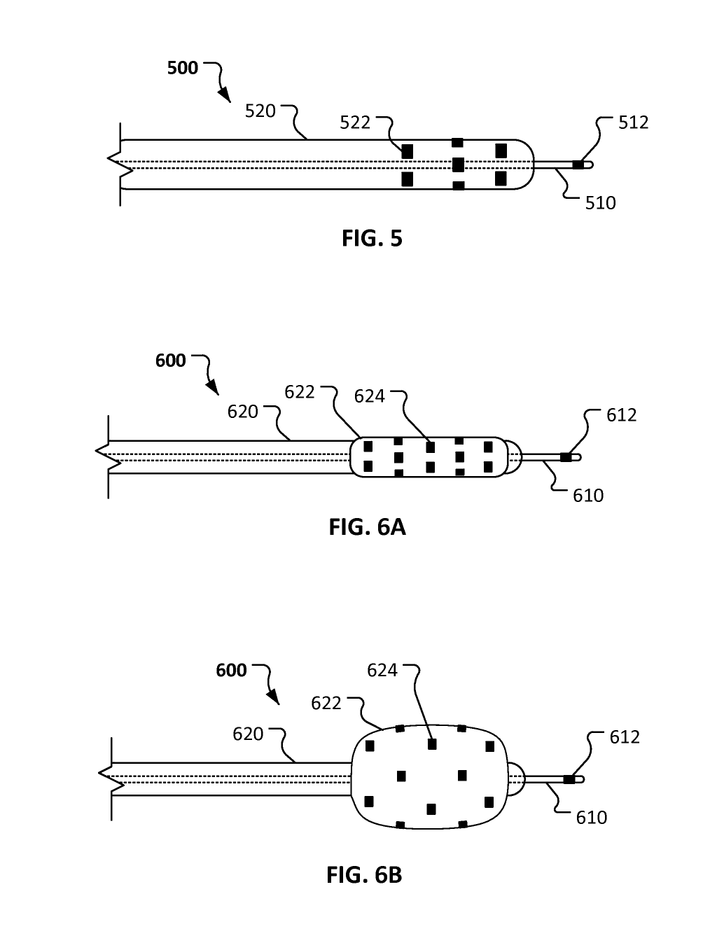

With reference to FIG. 5, a combination pacing/ablation and pressure-sensing device 500 can include a pressure wire 510 and a pacing/ablation catheter 520. Pressure wire 510 is slidably disposed within a lumen of pacing/ablation catheter 520. In other words, pacing/ablation catheter 520 is configured for over-the-wire delivery, and pressure wire 510 can be used as the guidewire and for taking pressure measurements.

Pressure wire 510 includes one or more pressure transducers 512. Pacing/ablation catheter 520 is configured to deliver pacing/ablation energy. Various types of pacing/ablation energy can be used. In the depicted embodiment, pacing/ablation catheter 520 includes multiple RF electrodes 522 as pacing/ablation energy delivery devices. In some implementations, a spacing of about at least 2 cm between the pacing/ablation electrodes 522 and pressure transducer 512 is maintained when combination pacing/ablation and pressure-sensing device 500 is in use.

With reference to FIGS. 6A and 6B, a combination pacing/ablation and pressure-sensing device 600 can include a pressure wire 610 and a pacing/ablation catheter 620. Pressure wire 610 is slidably disposed within a lumen of pacing/ablation catheter 620. As a result, pacing/ablation catheter 620 is configured for over-the-wire delivery, and pressure wire 610 can be used as the guidewire and for taking pressure measurements.

Combination pacing/ablation and pressure-sensing device 600 is configured with an expandable member 622 that includes pacing/ablation electrodes 624. In the depicted embodiment, expandable member 622 is a balloon. However, expandable member 622 can also be other types of expandable members as well.

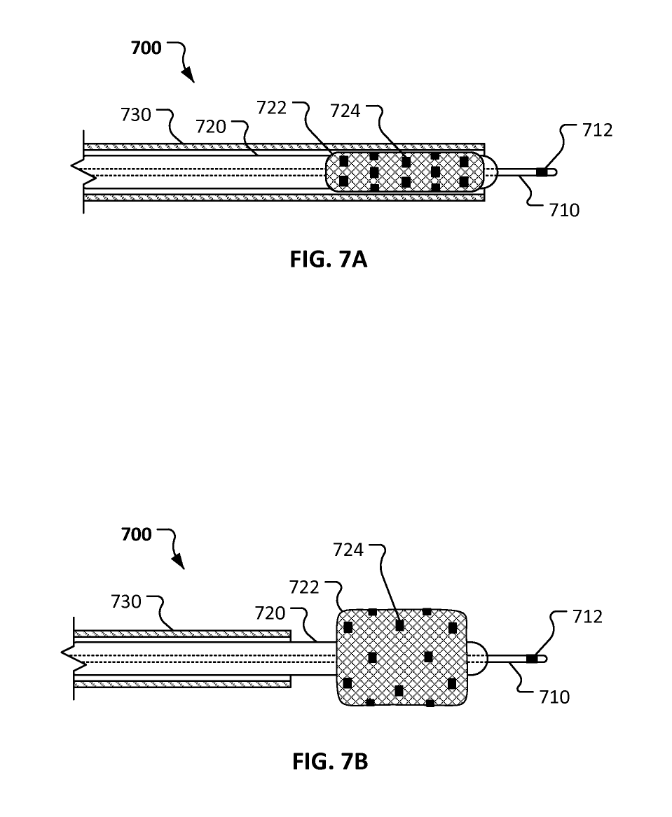

With reference to FIGS. 7A and 7B, another embodiment of a combination pacing/ablation and pressure-sensing device 700 can include a pressure wire 710 and a pacing/ablation catheter 720. Combination pacing/ablation and pressure-sensing device 700 can be configured for delivery using a sheath 730. Pressure wire 710 is slidably disposed within a lumen of pacing/ablation catheter 720. That is, pacing/ablation catheter 720 is configured for over-the-wire delivery, and pressure wire 710 can be used as the guidewire and for taking pressure measurements.

Combination pacing/ablation and pressure-sensing device 700 is configured with an expandable member 722 that includes pacing/ablation electrodes 724. In the depicted embodiment, expandable member 722 is a self-expandable stent-like frame. In some embodiments, expandable member 722 is comprised of nitinol. However, other materials such as, but not limited to, stainless steel can be used in other embodiments.

In some embodiments, the same principles relating to the devices and methods described above can be used by combining the devices with ultrasound catheters, cryoablation catheters, catheters placed in the renal pelvis, urinary bladder, ureters, or on retrograde canulation urinary tract catheters.

The devices and methods provided herein can also be performed or used for procedures that are used to affect local renal blood flow such as ablation or defibrillation of the kidneys, and for/with procedures using ultrasonic vibrations delivered from an external source, an internal source such as the renal vein or artery, or via the urinary system.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A transcatheter deliverable medical device system comprising:
a delivery sheath;
a mapping and ablation energy delivery device that is disposed on the distal end portion of a catheter and that is configured to expand to make contact with a vascular tissue of a patient when the mapping and ablation energy delivery device is not constrained by the delivery sheath; and
one or more pressure wires that are configured to extend from the delivery sheath and distally beyond the mapping and ablation energy delivery device such that the one or more pressure wires can be placed within one or more sub-branches of the vascular tissue, wherein the one or more pressure wires each include one or more pressure transducers that are configured to provide a signal indicative of a blood pressure at the location of the one or more pressure transducers.

2. The system of claim 1, wherein the mapping and ablation energy delivery device comprises a self-expandable member and one or more electrodes.

3. The system of claim 2, wherein the mapping and ablation energy delivery device comprises a nitinol member and a plurality of electrodes.

4. The system of claim 2, wherein the mapping and ablation energy delivery device comprises a balloon member and a plurality of electrodes.

5. A method for performing a renal denervation procedure, the method comprising:
delivering, to a renal artery, a transcatheter deliverable medical device system comprising:
a delivery sheath;
a mapping and ablation energy delivery device that is disposed on the distal end portion of a catheter and that is configured to expand to make contact with the renal artery when the mapping and ablation energy delivery device is not constrained by the delivery sheath; and
one or more pressure wires that are configured to extend from the delivery sheath and distally beyond the mapping and ablation energy delivery device such that the one or more pressure wires can be placed within one or more sub-branches of the renal artery, wherein the one or more pressure wires each include one or more pressure transducers that are configured to provide a signal indicative of a blood pressure at the location of the one or more pressure transducers;
measuring, by the one or more pressure transducers, a baseline blood pressure;
delivering, by the mapping and ablation energy delivery device, stimulation energy to the renal artery;
measuring, by the one or more pressure transducers, a post-stimulation blood pressure;
comparing the baseline blood pressure to the post-stimulation blood pressure; and
delivering, by the mapping and ablation energy delivery device and based on the comparison of the baseline blood pressure to the post-stimulation blood pressure, ablation energy to the renal artery.

6. The method of claim 5, wherein the baseline blood pressure is lower than the post-stimulation blood pressure.

7. The method of claim 5, further comprising after delivering the ablation energy:
measuring, by the one or more pressure transducers, a post-ablation blood pressure;
delivering, by the mapping and ablation energy delivery device, additional stimulation energy to the renal artery;
measuring, by the one or more pressure transducers, a post-ablation post-stimulation blood pressure;
comparing the post-ablation blood pressure to the post-ablation post-stimulation blood pressure; and
delivering, by the mapping and ablation energy delivery device and based on the comparison of the baseline blood pressure to the post-stimulation blood pressure, additional ablation energy to the renal artery.

8. The method of claim 7, wherein the comparison of the post-ablation blood pressure to the post-ablation post-stimulation blood pressure indicates that the additional ablation energy can be effective for reducing the post-ablation post-stimulation blood pressure further.

9. A method for monitoring for emboli, the method comprising:
delivering to a cerebral vasculature of a patient one or more pressure-sensing wires that each include one or more pressure transducers that are each configured to provide a signal indicative of a blood pressure at the location of the one or more pressure transducers;
measuring, by the one or more pressure transducers, a baseline blood pressure;
installing an implantable medical device within the patient's vasculature;
measuring, by the one or more pressure transducers and during or after the installing an implantable medical device within the vasculature of the patient, an intra-procedure blood pressure; and
comparing the baseline blood pressure to the intra-procedure blood pressure to determine whether intravascular emboli have been generated by the installing of the implantable medical device.

10. The method of claim 9 further comprising, in response to determining that intravascular emboli have been generated by the installing of the implantable medical device, installing or repositioning an emboli protection device within the patient's vasculature.
